(19) United States
(12) Patent Application Publication
(10) Pub. No.: US 2011/0270327 A1
(43) Pub. Date: Nov. 3, 2011

(54) IMPLANT GUIDE AND METHOD OF USE

(75) Inventors: David BLAKEMORE, Warsaw, IN (US); Eric M. LUCAS, Columbia City, IN (US)

(73) Assignee: VOT, LLC, Warsaw, IN (US)

(21) Appl. No.: 13/044,162

(22) Filed: Mar. 9, 2011

Related U.S. Application Data
(60) Provisional application No. 61/312,094, filed on Mar. 9, 2010.

(51) Int. Cl. A61B 17/56

(52) U.S. Cl. 606/86 R

(57) ABSTRACT

An implant guide for preparing a bone surface that includes a handle with an attached finger holder and a template head coupled to the handle. The template head is shaped to approximate the outer perimeter of an implant. The implant guide also includes a central opening that has an aperture and hook for receiving a marking device. A relief is positioned adjacent to the aperture to facilitate the engagement between the implant guide and the marking device. The implant guide may include a medial cutting notch and anterior slot for facilitating the preparation of the bone surface. A surgical kit is also disclosed that includes a plurality of implant guides, each having a handle and attached template head. The kit also includes an enclosure that is configured to hold the plurality of implant guides and marking devices. Further disclosed is a surgical method for using the implant guide.

### CROSS-REFERENCE TO RELATED APPLICATIONS

This application is entitled to the benefit of pending U.S. Provisional Patent Application Ser. No. 61/312,094 filed on Mar. 9, 2010, the disclosure of which is included by reference herein in its entirety.

### TECHNICAL FIELD

This invention relates generally to the field of surgical instruments, and more specifically to the field of artificial knee surgical instruments.

### BACKGROUND OF THE INVENTION

Surgical procedures often require the removal of a section or specific area of bone in order to accommodate an implant and bone cement. In these types of surgical procedures it is often necessary to have an instrument that is capable of providing proper sizing and positioning information to enable the operating surgeon to be able remove only a focal area of the bone without harming or damaging the remaining bone stock. The operating surgeon desires to limit any damage to the surrounding bone as it may compromise or weaken the remaining bone and subsequent bone-implant interface resulting in implant failure. However, it is often difficult to visualize the precise area of the bone to be resected and the amount to be resected. Therefore, it may be a challenge to precisely locate and determine the targeted area for bone removable that would be necessary for the implantation of a medical device.

Accordingly, it is recognized that there is a need for an implant guide which allows an operating surgeon to easily and rapidly determine the target position of the implant and the amount of bone to be removed without full visualization of the target bone area.

### SUMMARY OF THE INVENTION

Advancement of the state of surgical instrumentation that are used to determine the size and position on a bone surface for an implant, and more specifically, when a bone resection is to be performed with limited visibility for the implantation of a medical device is believed to be desirable. One example of an embodiment of the invention that satisfies the need for improvements is an implant guide used for determining the size and position of an implant on a bone surface that includes a handle and a template head that is attached to the handle. The template head is configured to approximate the outer shape of the implant. The template head permits the surgeon user to, among other things, determine the appropriate size of the implant to be used, the position of the implant and facilitate locating a marking device to be used as a reference point for a subsequent bone resection.

The present invention provides, in one aspect, an implant guide for preparing a bone surface that includes a handle and a template head that is attached to the handle and is configured to approximate or match an outer shape of an implant.

The present invention provides, in a further aspect, a surgical kit that includes an enclosure to retain a plurality of implant guides that each include a handle and template head that is sized to approximate a shape of an implant. Also included in the kit is at least one marking device.

The present invention provides, in another aspect, a surgical method of using the implant guide that may include the step of making a skin incision to expose a bone. The method may include the further step of placing the implant guide on a bone surface. The method may also include the step of positioning the implant guide in a location to expose a sufficient amount of bone around an outer perimeter of the implant guide. The method may further include the steps of securing the implant guide to a portion the bone surface with at least one fixation device and inserting a marking device through the implant guide and into the bone surface. The method may further still includes the step of removing the implant guide from the bone surface.

Other additional features, benefits, and advantages of the present invention will become apparent from the following drawings and descriptions of the invention. Other embodiments of the invention are described in detail herein and are considered a part of the claimed invention.

### BRIEF DESCRIPTION OF THE DRAWINGS

The subject matter which is regarded as the invention is particularly pointed out and distinctly claimed at the end of the specification. The foregoing and other objects, features, and advantages of the invention are apparent from the following detailed description taken in conjunction with the accompanying drawings in which:

FIG. 1 is a top view of one embodiment of an implant guide, in accordance with an aspect of the invention;

FIG. 2 is a top view of an alternative embodiment of the implant guide of FIG. 1, in accordance with an aspect of the invention;

FIG. 3A is a perspective view of the implant guide of FIG. 1, showing the template head configured to approximate the outside shape of an implant placed over a bone surface, in accordance with an aspect of the invention;

FIG. 3B is a perspective view of the implant guide of FIG. 1, showing the template head placed over a bone surface with a fixation device placed through a hole in the template head and into the bone, in accordance with an aspect of the invention;

FIG. 3C is a perspective view of the implant guide of FIG. 1, showing the template head placed over a bone surface with a fixation device placed through holes in the template head and into the bone and a marking device placed through the center opening of the template head and into the bone, in accordance with an aspect of the invention;

FIG. 3D is a perspective view of the implant guide of FIG. 1, showing the template head being withdrawn from the bone, the fixation device removed from the template head and the bone, with the marking device remaining in the bone, in accordance with an aspect of the invention;

FIG. 4 is a perspective view of the implant guide of FIG. 1, in accordance with an aspect of the invention; and

FIG. 5 is a surgical method for using an implant guide.

### DETAILED DESCRIPTION FOR CARRYING OUT THE INVENTION

Generally stated, disclosed herein is an implant guide and a surgical kit. Further, disclosed herein is a surgical method of using the implant guide determine the proper implant size and position on the bone surface. Any alterations and further modifications in the described embodiments, and any further applications of the principles of the invention as described herein are contemplated as would normally occur to one skilled in the art to which the implant guide invention relates.

In this detailed description and the following claims, the words proximal, distal, anterior, posterior, medial, lateral, superior and inferior are defined by their standard usage for indicating a particular part of an implant guide or surgical opening according to the relative disposition of the implant guide, surgical opening or directional terms of reference. For example, “proximal” means the portion of the implant guide positioned nearest the torso while “distal” indicates the part of the implant guide farthest from the torso. As for directional terms, “anterior” is a direction towards the front side of the body, “posterior” means a direction towards the back of the body, “medial” means towards the midline of the body, “lateral” is a direction towards the sides or away from the midline of the body, “superior” means a direction above, and “inferior” means a direction below another object or structure.

As used herein the terms “implant guide” and “guide” may be used interchangeably as they essentially describe the same type of surgical instrument.

Generally stated, disclosed herein is an implant guide for use in surgical procedures. The implant guide shown is intended for example purposes only and generally includes a handle and a template head configured to approximate the outer circumferential shape of a medical implant. Throughout the disclosure, the template head is described as approximating the shape and area of a medical implant.

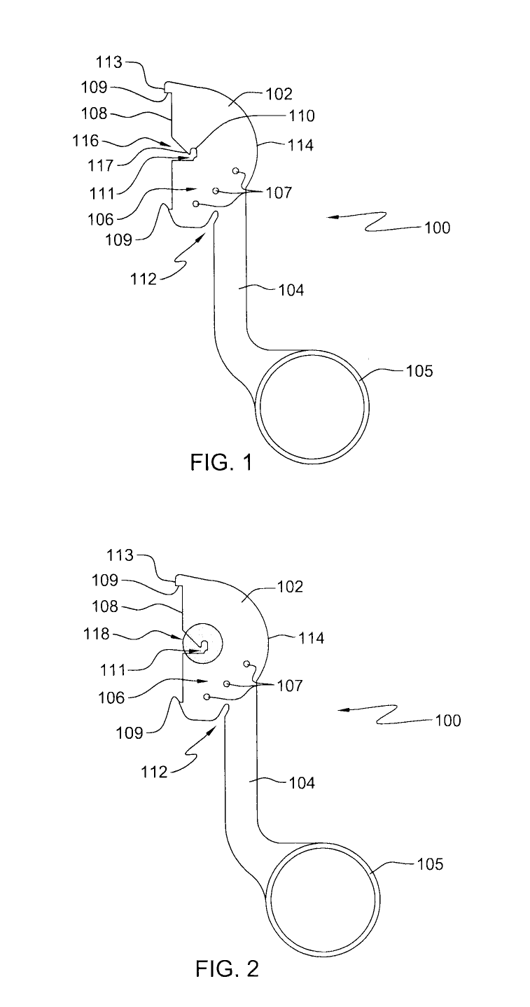

FIG. 1 shows a top view of an implant guide 100 that includes a template head 102 configured to approximate the outer shape of an implant, which for example purposes in FIG. 1 is a unicondylar tibial knee implant, although other implant shapes may be used. Implant guide 100 also includes a handle 104 attached to template head 102. In the embodiment of FIG. 1, a means for securing the template head 106 is shown as a plurality of apertures or holes 107 formed in template head 102. An outer perimeter 114 of template head 102 is shown generally as a D-shaped for example purposes. However, alternative embodiments of template head 102 may have a variety of shapes to correspond to an implant shape and size. For example, outer perimeter 114 may be generally a circle, a half circle, an ellipsis, an oval, a square, a rectangle or other polygonal or otherwise irregular shape. Further, outer perimeter 114 of template head 102 may be of different sizes to approximate the various sizes of implants that implant guide 100 is replicating. Use of alternative sizes of template head 102 may be dependent on the size of the implant, the size of the bone, the nature of the operation as well as other clinical considerations.

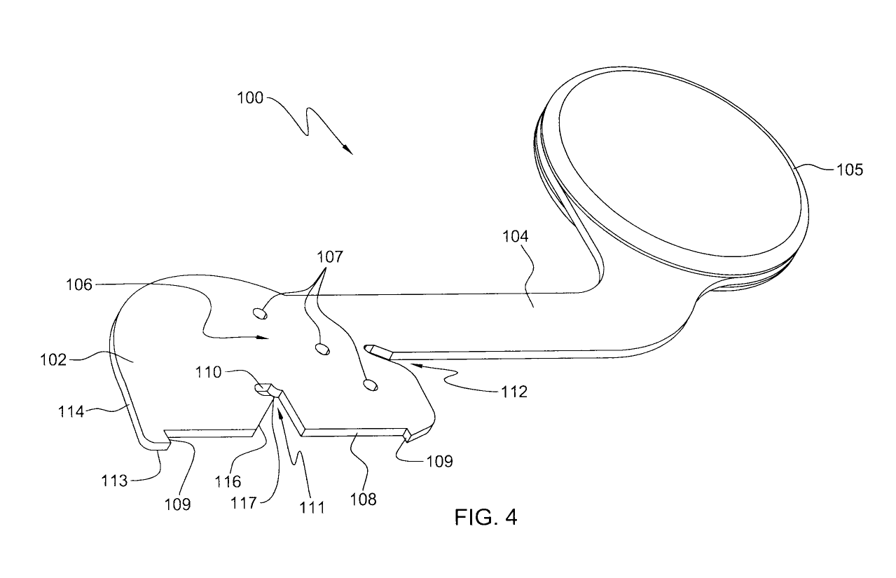

FIGS. 1 and 4 also shows template head 102 having a slot 112 in the anterior aspect of outer perimeter 114. Slot 112 is configured to receive a bone chisel or other cutting device that is used to perforate the bone surface while implant guide 100 is secured to the bone surface. Slot 112 may also be positioned in different locations along outer perimeter 114 to accommodate other implant designs or alternatively, be omitted from template head 102 entirely.

As seen in FIGS. 1 and 4, notch 108 extends partially along the medial side 113 of outer perimeter 114. Notch 108 is recessed to accommodate a cutting tool that is used to perforate the bone surface. Two stops 109 are seen at the posterior edge of medial side 113 and the anterior edge of medial side 113. Stops 109 function to provide visual and tactile feedback to the surgeon user of the maximum anterior-posterior length of the cut into the bone surface that is created along notch 108. For the implant guide 100 shown in FIGS. 1 and 4, the cut is typically oriented in the area of the tibial spine that may be cut or removed.

Further as seen in FIGS. 1 and 4, positioned in the center aspect of medial side 113 is relief 116. As shown, relief 116 is generally triangular, however, it is contemplated that relief 116 may be, for example purposes other geometric configurations such as angular, linear, square, circular, oval, or polygonal. Relief 116 is sized and configured to allow access to the central opening 111. Relief 116 extends from medial side 113 to an aperture 110.

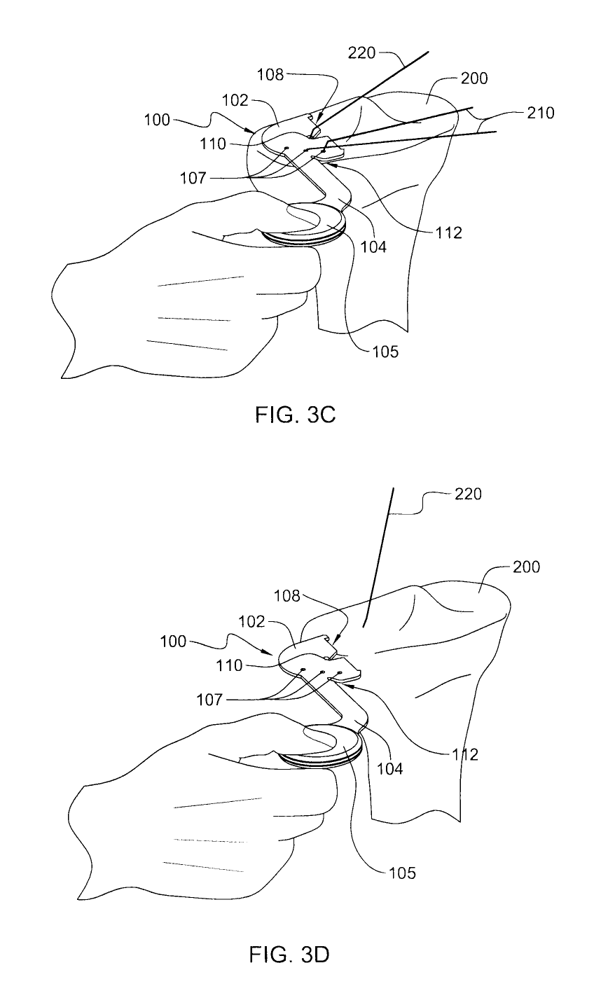

As shown in FIGS. 1 and 4, central opening 111 includes aperture 110 which is generally circular, but other configurations may be used including, for example purposes, square, rectangular, circular, oval, and polygonal. Aperture 110 is positioned approximately in the center of template head 102 so as to assist the surgeon user with positioning implant guide 100 in the center of the target bone surface. Aperture 110 may be sized to receive a marking device 220 (See FIG. 3C). Marking device 220 may be in the form of a k-wire, pin, drill bit, screw or needle. Central opening 111 also includes a hook 117 located at the medial aspect or exit point of aperture 110. As exhibited in FIGS. 1 and 4, hook 117 functions to capture marking device 220 within aperture 110 to assist the surgeon user in retaining the proper positioning of marking device 220 while it is inserted. Hook 117 acts to keep marking device 220 from sliding or skiving away from aperture 110 when being inserted though implant guide 100 and into bone 200.

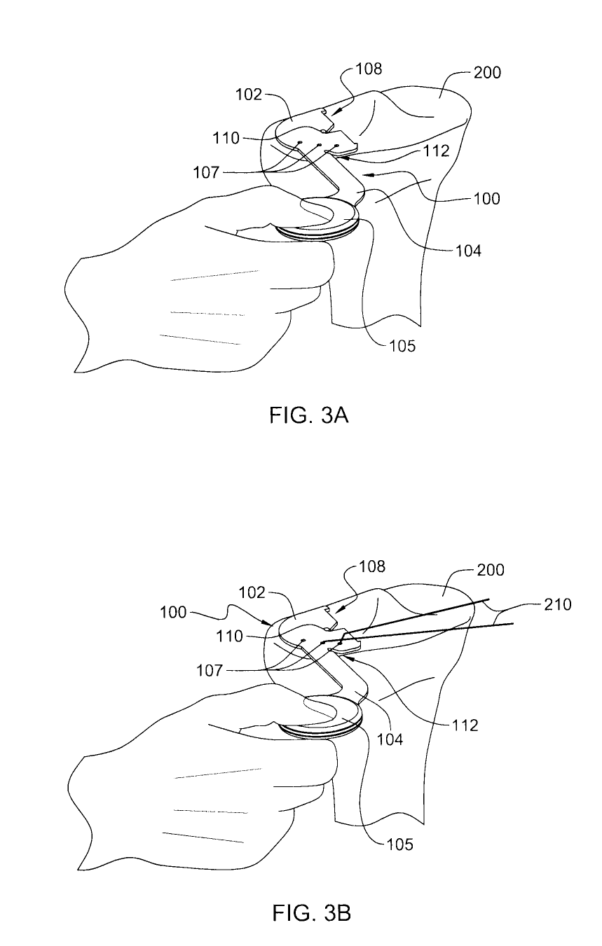

FIG. 2 shows an alternative embodiment of implant guide 100 that includes a membrane 118. As seen, membrane 118 is positioned to partially cover template head 102, relief 116 and central opening 111. Membrane 118 may extend to fully cover aperture 110. Membrane 118 is shown as having a circular construct, but other shapes, may be used including, for example purposes, square, rectangular, circular, oval, and polygonal. Membrane 118 may be fabricated from rubber, silicon, or another flexible material that allows for penetration and the capture of marking device 220 so as to allow the surgeon user to insert marking device 220 into membrane 118 before implant guide 100 is inserted through the incision and placed onto the bone surface.

FIGS. 1 and 4 show a means for securing template head 102 to the bone surface 106 that includes a plurality of holes 107. For example purposes, holes 107 are shown in FIGS. 1, 3A and 4 to be placed along the anterior aspect of template head 102. Holes 107 may be positioned in other locations to address anatomic requirements of other target bones in which implant guide 100 may be used. Holes 107 are sized to receive pins, wires, drill bits or other temporary fixation devices 210 that are used to secure implant guide 100 to the bone surface prior to the insertion of marking device 220.

FIGS. 3B and 3C show fixation devices 210 following insertion into holes 107. Alternative embodiments of means for securing the template head to the bone surface 106 are not shown but may include spikes or pins which may extend from the inferior or bone contacting surface of template head 102. Template head 102 may be fixed to the bone by applying pressure on the superior surface of template head 102, thereby pushing the pins or spikes into the bone surface. Another alternative embodiment of means for securing the template head to the bone surface 106 could be in the form of a temporary adhesive which is applied to the inferior surface of template head 102 or another surface treatment that results in template head 102 sticking to the bone surface following the application of pressure.

In the embodiment shown in FIGS. 1, 2 and 4, handle 104 and finger holder 105 is shown as being relatively coplanar with template head 102. Handle 104 is seen as extending in a generally anterior direction from template head 102 and offset from slot 112 to allow for accessibility. Finger holder 105 is further offset to the lateral side to remain clear of the incision. As seen in FIG. 3A, finger holder 105 is shaped to provide for an ergonomic fit between the finger and thumb of the surgeon user outside of the joint space, while template head 102 remains within the wound and on the bone surface. Handle 104 may also extend in a generally lateral direction in alternative embodiments, if the skin incision used requires an orientation in such a direction. Although not shown, handle 104 could extend in more than one direction, for example, in both an anterior and inferior or superior direction with the direction and angulation of handle 104 being varied based on the surgical procedure and approach involved. As shown in FIGS. 1 and 4, only one handle 104 is attached to template head 102, it is understood that for certain clinical situations more than one handle may be desirable to assist with stabilizing template head 102 on the bone surface.

As exhibited in FIGS. 1 and 4, handle 104 is integral with template head 102. A modular handle may be used that would allow the surgeon user to attach various sized and oriented handles to template head 102. Common detachable securement mechanisms would be used to couple template head 102 to handle 104 including but not limited to, a pin lock, spring clamp and snap fit. The surgeon user may choose to keep handle 102 in place throughout the surgical procedure or remove it once template head 102 is secured in position on the bone surface.

In FIG. 4, implant guide 100 is shown as being generally planar, and extending in only two dimensions. To facilitate closeness to the target bone surface, template head 102 may be concave or convex. Concave and convex surface geometries for template head 102 may allow the surgeon user to more closely approximate the fit and positioning of template head 102 to allow for optimizing these assessments.

Implant guide 100 may be fabricated from a no-brittle, flexible and durable plastic material. Examples of suitable materials for the construct include, but are not limited to, nylon, an acetal copolymer, an acetal homopolymer, polyethelene, and PEEK. Implant guide 100 may be fabricated from of another biocompatible, autoclavable, and sterilizable material, such as, for example, surgical steel or hard plastic.

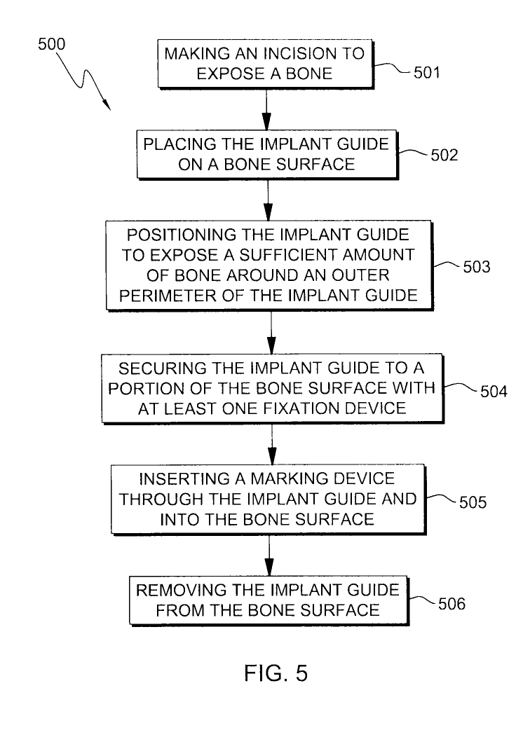

The surgical method for using the implant guide is also disclosed herein and shown in FIG. 5. This description of the surgical procedure should be understood to encompass all possible embodiments of the implant guide previously discussed. The surgical method of using an implant guide 500 includes, generally, the step of making an incision over the target joint to expose the bone 501. The next step involves determining the appropriate sized implant guide with a device that establishes the posterior margin of the tibia along the center spine structure. This tool is necessary as visualization of the posterior portion of the tibia is difficult. The template head size is typically chosen based off of this initial measurement.

As seen in FIGS. 3A and 5, the next step of the method is to place the implant guide on a bone surface 502 which, for example purposes, may be the proximal tibia. An additional step may be to locate the implant guide to ensure that a sufficient amount of bone is exposed around the outer perimeter of the implant guide 503. Depending upon the quality of bone stock, sufficient amounts of bone may range from 2 to 6 mm.

FIGS. 3B and 5 exhibit the further step of securing the implant guide to a portion of the bone surface with at least one fixation device 504. FIG. 3B shows two fixation devices 210 being used, but depending on the surgeon user preference a range of 1 to 3 fixation devices may be used. In an alternative method, fixation devices may be replaced by other common medical securing devices or fasteners. It is also contemplated that the inferior surface of template head may be coated with a friction-like material which temporarily attaches the template head to the bone surface. In still another alternative, the template head may be attached to the bone using pins or spikes that extend from the inferior surface of the template head.

FIG. 3C shows that upon securement of the implant guide, the method may include the step of inserting a marking device through the aperture of the central opening that is positioned in the central portion of the template head, which corresponds to the center of the intended implant, and into the bone 505. The marking device may be in the form of a pin, k-wire, drill bit or screw.

Using the alternative embodiment of implant guide that is shown in FIG. 2, the marking device may be inserted though a membrane before the implant guide, and more specifically the template head, is placed in close proximity to the bone. This may facilitate the insertion of the marking device into the bone.

Subsequent to the securement of the implant guide and placement of the center marking device, the surgeon user may cut along the medial edge, more specifically the notch and also along the anterior slot to accommodate the various structural elements of the implant that may be present. Following the resection of bone medially and anteriorly, the at least one fixation device may be removed leaving the centralized marking device in place.

As seen in FIG. 3D (and described in FIG. 5), the implant guide is removed using a slight posterior directed force to disengage the marking device from the aperture and hook, leaving the marking device in place in the bone and allowing for the removal of the implant guide from the bone surface and incision 506. The intact marking device may now be used a reference point for further bone resection procedures, including being attached to a burring or cutting device via a tether mechanism. Once marking device is attached to the cutting or burring device via the tether, the cutting device can be moved in a semicircular fashion at a set distance from the marking device. This will result in an inlayed or recessed channel that is shaped like the outside configuration of the implant guide and corresponding implant. A rim of bone ranging from 2 to 6 mm is created around the outer periphery of the proximal tibia that mirrors the implant size and corresponds to the chosen implant guide. The rim will act to protect the implant and also capture inserted bone cement

A surgical kit is also disclosed, with the surgical kit including among other items, a plurality of implant guides 100, each of the implant guides having the structural aspects (template head and handle) as described above and for brevity sake will not be described again. The kit may further include an enclosure for housing the plurality of implant guides, various marking devices, a plurality of fixation devices, bone cutters/chisels and saw blades. If the implant guide includes a modular handle, various sized and angled handles may also be included in the surgical kit.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. An implant guide for preparing a bone surface, the implant guide comprising:
a handle; and
a template head coupled to the handle and configured to approximate an outer shape of an implant.

2. The implant guide of claim 1, wherein the template head comprises an outer perimeter having an anterior to posterior dimension and a medial to lateral dimension, wherein the anterior to posterior dimension approximates the anterior to posterior dimension of the implant.

3. The implant guide of claim 2, wherein the outer perimeter is substantially D-shaped to facilitate positioning of the template head when visualization of the bone surface is restricted.

4. The implant guide of claim 1, wherein the template head further comprising a central opening adapted to receive a marking device.

5. The implant guide of claim 4, wherein the central opening is configured to restrict movement between the template head and the marking device.

6. The implant guide of claim 4, wherein the central opening comprises an aperture and a hook.

7. The implant guide of claim 6, wherein the central opening further comprises an angled relief adjacent to the aperture to facilitate engagement with the marking device.

8. The implant guide of claim 4, wherein the central opening comprises an aperture, a hook and a membrane, the membrane configured to cover the aperture and operatively hold the marking device in a set position.

9. The implant guide of claim 8, wherein the membrane comprises at least one of rubber, silicon and a polymer.

10. The implant guide of claim 1, wherein the handle is offset from the template head to permit visualization of the bone surface.

11. The implant guide of claim 1, further comprising at least one slot, the at least one slot adapted to receive a cutting tool.

12. The implant guide of claim 11, wherein the at least one slot extends from an anterior aspect of the template head.

13. The implant guide of claim 1, further comprising a notch configured to receive a cutting tool.

14. The implant guide of claim 13, wherein the notch is positioned along a medial side of the template head to facilitate the resection of the bone surface.

15. The implant guide of claim 1, further comprising means for securing the template head to the bone surface.

16. The implant guide of claim 15, wherein the means for securing the template head to the bone surface comprises at least one through hole and at least one fixation device inserted into the at least one through hole.

17. The implant guide of claim 15, wherein the means for securing the template head to the bone surface comprises at least one of a plurality of spikes and a plurality of pins extending from an inferior surface of the template head.

18. A surgical kit comprising:
a plurality of implant guides recited in claim 1.

19. The surgical kit of claim 18, further comprising an enclosure adapted to retain the plurality of implant guides and at least one marking device.

20. A surgical method for using an implant guide, the method comprising:
making a skin incision to expose a bone;
placing the implant guide on a bone surface;
positioning the implant guide to expose a sufficient amount of bone around an outer perimeter of the implant guide
securing the implant guide to a portion of the bone surface with at least one fixation device;
inserting a marking device through the implant guide and into the bone surface; and
removing the implant guide from the bone surface.
